(19) United States
(12) Patent Application Publication
(10) Pub. No.: US 2009/0192509 A1
(43) Pub. Date: Jul. 30, 2009

(54) Electrosurgical system

(76) Inventors: Richard J. Curtis, Newport (GB); Michael D. Newton, Newport (GB)

Correspondence Address: NIXON & VANDERHYE, PC, 901 NORTH GLEBE ROAD, 11TH FLOOR, ARLINGTON, VA, 22203, US

(21) Appl. No.: 12/318,969

(22) Filed: Jan. 13, 2009

Related U.S. Application Data
(60) Provisional application No. 61/006,720, filed on Jan. 29, 2008.

(30) Foreign Application Priority Data
Jan. 14, 2008 (GB) 0800593.6

(51) Int. Cl. A61B 18/14

(52) U.S. Cl. 606/42

(57) ABSTRACT

An electrosurgical system includes an electrosurgical generator (1) having at least one source of radio frequency (RF) power, at least one output connection (2, 3, 4), and a switch input connection (25). At least one electrosurgical assembly is connected to the generator (1) via a respective output connection (2, 3, 4), each electrosurgical assembly including an electrosurgical instrument (5, 6, 7), and a cable (8, 9, 10), connecting the electrosurgical instrument to a respective output connection. The electrosurgical system also includes at least first and second switch assemblies, each adapted to control the operation of the electrosurgical assemblies, and each including a switch module (11, 11′, 17, 18, 19), a cable (12, 12′, 20, 21, 22), and a switch connector (38, 38′, 39, 40, 41). An adapter unit in the form of an adapter cable (30) is provided, the adapter cable including a first connector (31) adapted to interconnect with the switch input connection (25) of the generator (1), and at least second and third connectors (36, 37), each of the second and third connectors being adapted to interconnect with the switch connector of the first and second switch assemblies.

### BACKGROUND OF THE INVENTION

This invention relates to an electrosurgical system including an electrosurgical generator, and one or more electrosurgical instruments for use therewith.

The majority of electrosurgical generators are designed to have only one instrument attached to the generator at any one time. However, there is an increasing trend, with ever more sophisticated electrosurgery systems, to have multiple instruments connected to the generator at any one time. U.S. Pat. Nos. 5,342,356 and 6,508,809 are two examples of electrosurgical systems in which multiple instruments can be connected to a single generator. These patents describe how shaped-connectors, color-coding and distinct symbols can be used to assist with the connection of these multiple instruments, and to ensure that the correct instrument is connected to the appropriate output of the generator.

Another example of an electrosurgery system with multiple instruments connected to a single generator is US application 2004/0215131. Like most of these electrosurgical generators, this application describes a system that only allows for the operation of one of the attached instruments at any one time. Handswitches are present on the electrosurgical instruments, and the pressing of any of the handswitches immediately makes that instrument the “active” instrument.

With more sophistication being introduced to electrosurgical systems, the control of the instruments becomes more complex. It is an object of the present invention to provide an improved electrosurgical system in which various electrosurgical instruments can be controlled more easily.

### SUMMARY OF THE INVENTION

Accordingly, an electrosurgical system is provided comprising;
i) an electrosurgical generator comprising;
   a) at least one source of radio frequency (RF) power,
   b) at least one output connection, the or each output connection being such that at least a first electrosurgical assembly can be connected to the generator; and
   c) a switch input connection;
ii) at least one electrosurgical assembly comprising an electrosurgical instrument and a cable connecting the electrosurgical instrument to a respective output connection;
iii) at least first and second footswitch assemblies, each adapted to control the operation of the respective electrosurgical assembly, and each comprising a switch module, a cable and a switch connector; and
iv) an adapter unit comprising a first connector adapted to interconnect with the switch input connection of the generator, and at least second and third connectors, the second and third connectors being adapted to interconnect respectively with the switch connectors of the first and second footswitch assemblies.

The adapter unit, with a first connector for connection with the generator, and two or more further connectors for connecting with the switch assemblies, provides more options for the control of the electrosurgical system. This allows for two footswitch assemblies to be connected to the electrosurgical generator. It is nowadays common for two or more surgeons to be present during a surgical procedure, especially during some of the more complex and/or time-consuming surgery. Often, each of the surgeons is holding an electrosurgical instrument, and the surgeons take turns to perform parts of the procedure. With only a single footswitch, the footswitch is often moved back and forth under the operating table, as a surgeon on each side of the patient needs to activate his/her electrosurgical instrument. By way of the present invention, a surgical system can be provided in which each surgeon has a footswitch, or at the very least a footswitch is provided on each side of the operating table.

Published application WO2005/048860 describes a “Y-Cable” used with an electrosurgical generator. However, the Y-Cable of WO2005/048860 is used to allow a single RF output, typically situated on the front of the generator, to drive two electrosurgical instruments. This is fundamentally different from the present invention which uses the adapter unit to allow multiple switch assemblies to be connected to the switch input connection, typically situated on the back of the generator.

In a preferred embodiment, there is further provided a handswitch mounted on one of the electrosurgical instruments, the handswitch comprising a switch module, a cable and a switch connector, the adapter unit further comprising a fourth connector adapted to interconnect with the switch connector of the handswitch. In this way, the surgeon can be presented with the option of controlling an electrosurgical instrument either by a conventional footswitch or by the handswitch mounted on the instrument. The provision of the adapter unit allows for both the footswitches and the handswitch to be connected to the single switch input connection provided on the electrosurgical generator.

The adapter unit typically comprises a cable unit comprising only cables and connectors. This is simpler than having an adapter box, containing circuitry and other components. In its simplest form, the adapter unit can have a first connector for the generator, and second and third connectors for the footswitch assemblies (typically a “Y-Cable”).

In a typical arrangement, there are at least first and second electrosurgical assemblies respectively connected to first and second output connections of the generator. Conceivably, each of the electrosurgical assemblies is provided with a handswitch, and the adapter unit is provided to allow both handswitches to be simultaneously connected to the electrosurgical generator. Each handswitch can be used to send signals to the electrosurgical generator, to cause any particular instrument to be used. One protocol for determining the command structure for the different electrosurgical instruments is described in our co-pending GB patent application 0800772.6.

Therefore, according to an alternative aspect of the invention, there is provided an electrosurgical system comprising:
i) an electrosurgical generator comprising:
   a) at least one source of radio frequency (RF) power,
   b) at least two output connections, the output connections being such that at least two electrosurgical assemblies can be connected to the generator; and
   c) a switch input connection;
ii) at least two electrosurgical assemblies, each comprising an electrosurgical instrument and a cable connecting the electrosurgical instrument to a respective output connection;
iii) at least first and second handswitch assemblies, each adapted to control the operation of the respective electrosurgical assembly, and each comprising a switch module, a cable and a switch connector; and
iv) an adapter unit comprising a first connector adapted to interconnect with the switch input connection of the generator, and at least second and third connectors, the second and third connectors being adapted to interconnect respectively with the switch connectors of the first and second handswitch assemblies.

As before, the adapter unit can have first connector for the generator, and second and third connectors for the handswitch assemblies (typically a “Y-Cable”). Alternatively, where there are at least three handswitch assemblies, or two handswitch assemblies and a footswitch assembly, and the adapter unit has four or more connectors such that there are at least three connectors adapted to interconnect with the switch connectors of the three switch assemblies. This typically makes the adapter unit an “X-Cable”, with one connector for the generator, and three (or conceivably even more further) connectors for the handswitches of the electrosurgical instruments.

### BRIEF DESCRIPTION OF THE DRAWINGS

The invention will now be described in more detail, by way of example only, with reference to the accompanying drawings, in which;

FIG. 1 is a schematic view of an electrosurgical system constructed in accordance with the invention;

FIG. 2 is a plan view, partly in section, of an adapter unit forming part of the electrosurgical system of FIG. 1;

FIG. 3 is a schematic view of an electrosurgical system constructed in accordance with an alternative embodiment of the invention; and

FIG. 4 is a schematic view of an electrosurgical system constructed in accordance with a further embodiment of the invention.

### DESCRIPTION OF PREFERRED EMBODIMENTS

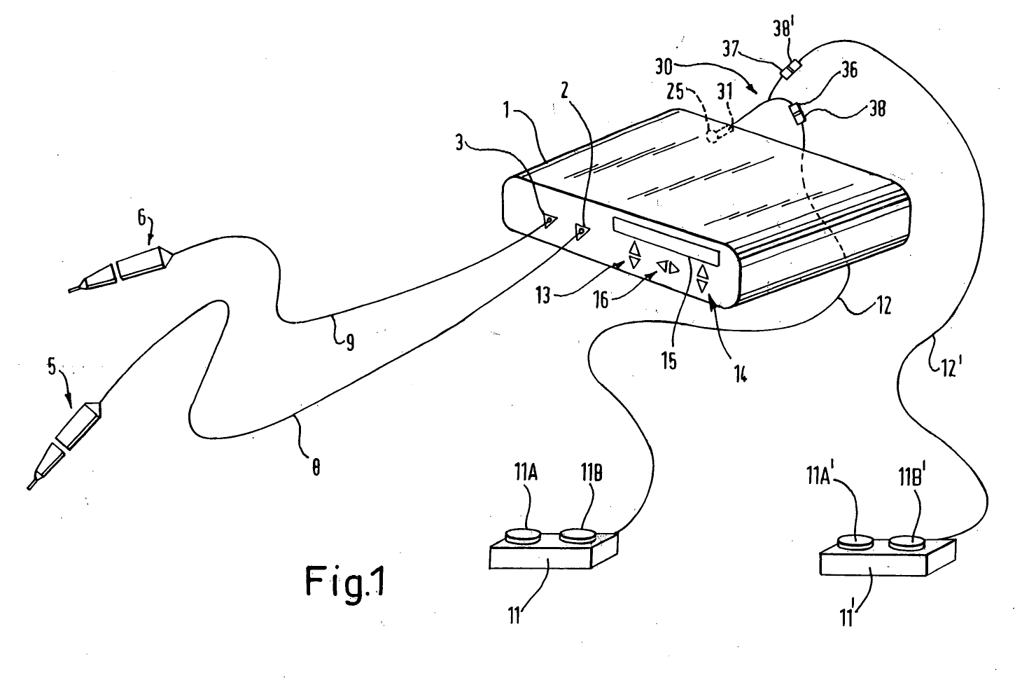

Referring to FIG. 1, a generator 1 has an output socket 2 providing a radio frequency (RF) output for an electrosurgical instrument 5 via a connection cord 8, and an output socket 3 for electrosurgical instrument 6 via connection cord 9. Activation of the generator 1 may be performed from a footswitch unit 11, as shown, connected to the generator via a footswitch connection cord 12, or from a second footswitch unit 11′ via a footswitch connection cord 12′. The footswitch unit 11 has two footswitch pedals 11A and 11B for selecting a coagulation mode and a cutting mode of the generator 1 respectively. Similarly, the footswitch unit 11′ has two footswitch pedals 11A′ and 11B′, also capable of directing the generator 1 to operate either in a coagulation mode or a cutting mode. The footswitch units 11 and 11′ may each have one or more additional buttons (not shown), for example for entering commands for changing the operating parameters of the generator 1. The generator front panel has push buttons 13 and 14 for respectively setting coagulation and cutting power levels, which are indicated in a display 15.

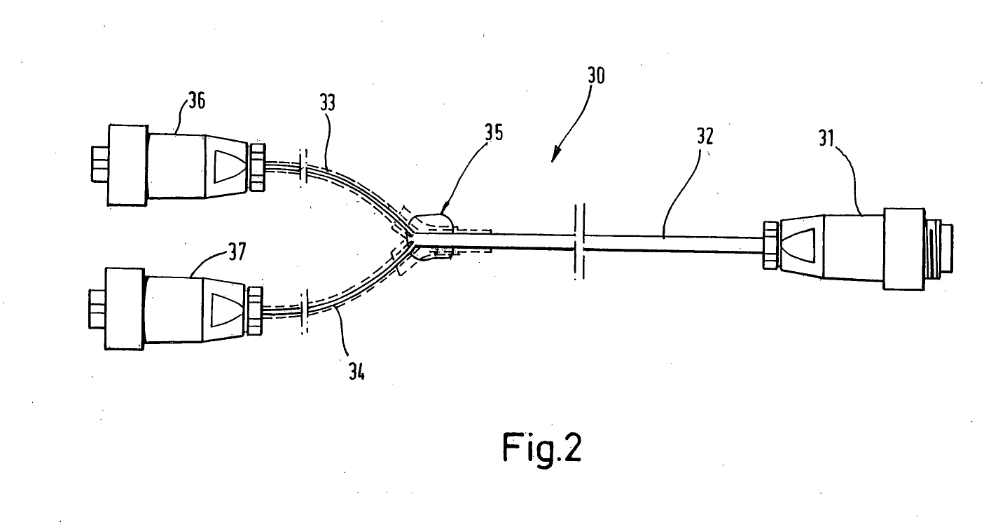

Both the footswitch connection cords 12 and 12′ are connected to a single switch socket 25 located on the rear of the generator 1. This is achieved by means of an adapter unit in the form of an adapter cable 30, which will now be described in more detail with reference to FIG. 2.

The adapter cable 30 comprises a generator plug 31 adapted to be received in the switch socket 25 on the back of the generator 1. Emerging from the plug 31 is a main cable 32, which divides into two sub-cables 33 and 34 at a Y-shaped division section 35. A first adapter cable socket 36 is present on the end of the sub-cable 33, while a second adapter cable socket 37 is present on the end of the sub-cable 34. The operation of the adapter cable will now be described.

Firstly, the adapter cable 30 is attached to the generator 1 by inserting the generator plug 31 into the switch socket 25. Then a footswitch plug 38 on the end of the footswitch connection cord 12 is attached to the first adapter cable socket 36. Finally, a second footswitch plug 38′ on the end of the footswitch cable 12′ is attached to the second adapter cable socket 37. The footswitch units 11 and 11′ are now both connected to the generator 1 as shown in FIG. 1.

In this way, commands can be given to the generator 1 to activate and control the operation of the instrument 5 by either the footswitch unit 11 or the footswitch unit 11′. Where there are two surgeons performing a surgical procedure, one using the instrument 5 and the other using the instrument 6, one surgeon can use the footswitch unit 11 and the other the footswitch unit 11′. In general, only one of instruments 5 and 6 may be activated at any one time, and one process for controlling and changing which instrument is the “active” instrument is described in our co-pending GB patent application 0800772.6. The changeover from one instrument to the other can also be carried out by means of the push buttons 16 on the generator 1, or by a sequence of signals sent from the footswitch units 11 and 11′. Typically, the electrosurgical system of FIG. 1 is used by two surgeons, one situated on each side of an operating table (not shown). With a single footswitch, it often seems that the footswitch has to be passed from one surgeon to the other. In contrast, the provision of the two footswitch units 11 and 11′ allows for one footswitch unit to be present on each side of the operating table, such that each surgeon can access a footswitch without the need for a footswitch to be moved or passed from one surgeon to the other, or for both surgeons to try and use a single footswitch unit in a central location.

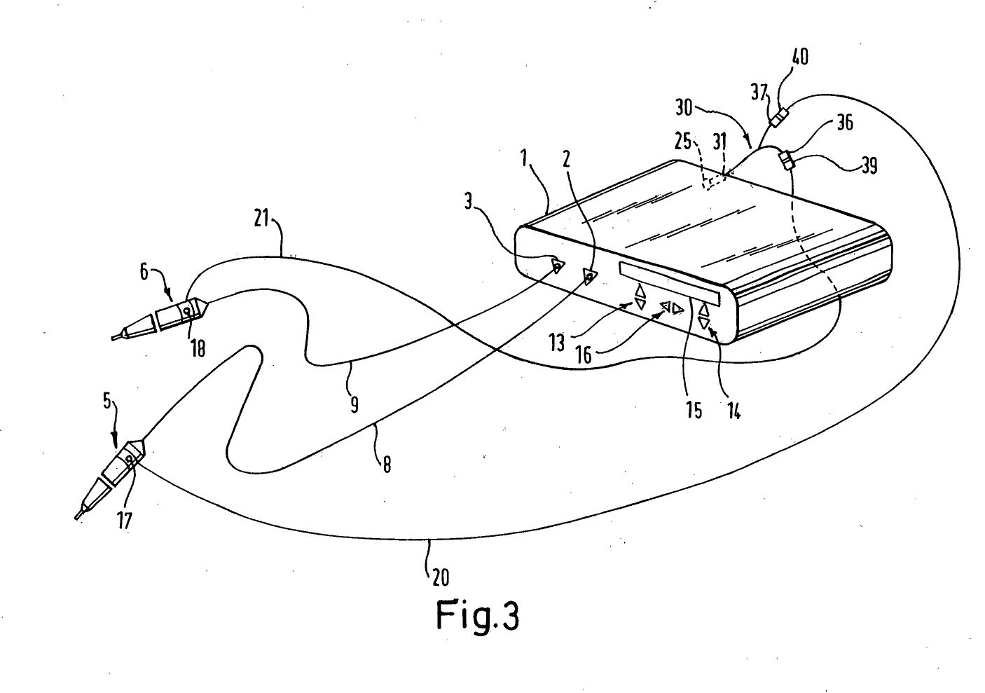

FIG. 3 shows an alternative arrangement in which the instruments 5 and 6 are each provided with a respective handswitch 17, 18. The handswitch 17 is mounted on the instrument 5, and sends commands to the generator 1 via a handswitch cable 20. Similarly, the handswitch 18 is mounted on the instrument 6, and sends signals to the generator 1 via a handswitch cable 21. The handswitch cable 20 terminates in a handswitch plug 39, while the handswitch cable 21 terminates in a handswitch plug 40. The plug 31 of the adapter cable 30 is inserted into the back of the generator 1 as before, and the handswitch plugs 39 and 40 are attached to the first and second adapter cable sockets 36 and 37. In this way, both the handswitches 17 and 18 can be used to control the electrosurgical instruments 5 and 6.

Where there are multiple instruments, as in FIG. 3, a system must be put in place to control the transfer of the operation of one instrument to another. Such a system is described in our co-pending GB patent application 0800772.6. In this, control of the instruments is either “pulled” from one instrument to another, or “pushed” from one instrument to another as described in more detail therein.

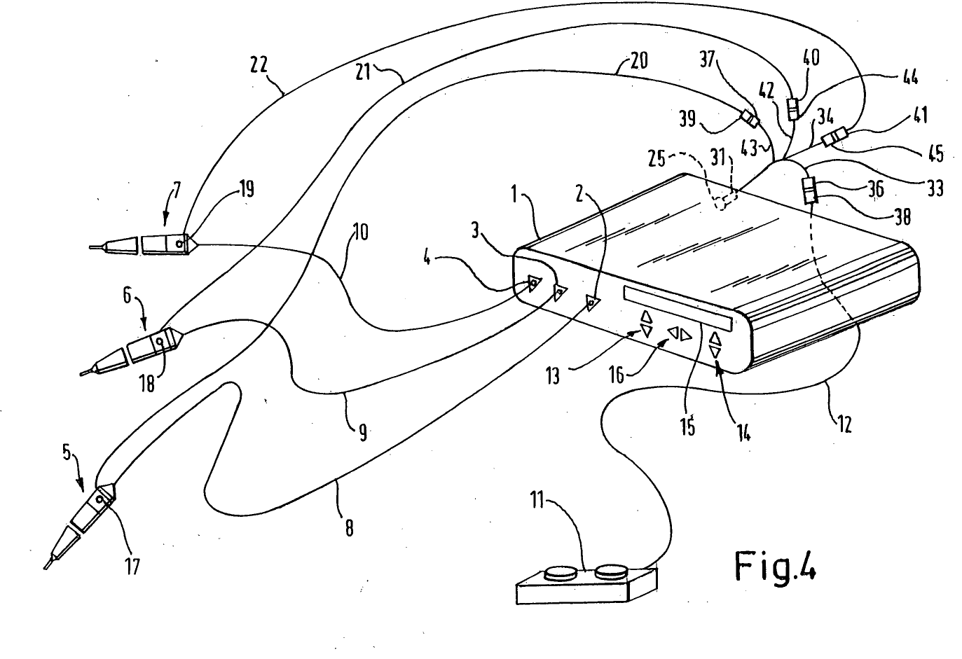

FIG. 4 shows an alternative system in which only one footswitch unit 11 is present, but three instruments 5, 6 and 7 are connected to the generator 1, the additional instrument 7 being connected by a connection cord 10 in an output socket 4. Furthermore, each of the instruments 5, 6 and 7 is provided with a handswitch 17, 18 and 19 respectively. The handswitch 17 has the handswitch cable 20 as previously described, the handswitch 18 has the handswitch cable 21 as previously described, and the handswitch 19 on the instrument 7 has a handswitch cable 22.

The cable 20 terminates in the handswitch plug 39 as previously described, the cable 21 terminates in the plug 40 as previously described, and the cable 22 terminates in a plug 41. The adapter cable 30 is of a different design, having four sub-cables 33, 34, 42 and 43, and four adapter cable sockets 36, 37, 44 and 45. The footswitch plug 38 is received in the adapter cable socket 36, to connect the footswitch unit 11 to the generator 1. Additionally, the handswitch plugs 39, 40 and 41 are received in the adapter cable sockets 37, 44 and 45, to connect the handswitches 17, 18 and 19 to the generator 1. In this way, the operation of the instruments 5, 6 and 7 can be controlled either by the footswitch unit 11, or by the handswitches 17, 18, 19 present on the instruments 5, 6, 7.

As before, a hierarchy must be established within the generator 1 to deal with commands from the handswitches 17, 18, 19 and the footswitch 11. The software hierarchy may be such that commands from one or other of the footswitch and the handswitches take priority over the other, or the generator 1 may be programmed to respond to whichever signals are received in turn.

What is claimed is:

1. An electrosurgical system comprising:
i) an electrosurgical generator comprising:
a) at least one source of radio frequency (RF) power,
b) at least one output connection, the or each output connection being such that at least a first electrosurgical assembly can be connected to the generator, and
c) a switch input connection;
ii) at least one electrosurgical assembly comprising an electrosurgical instrument and a cable connecting the electrosurgical instrument to a respective output connection;
iii) at least first and second footswitch assemblies, each adapted to control the operation of the respective electrosurgical assembly, and each comprising a switch module, a cable and a switch connector; and
iv) an adapter unit comprising a first connector adapted to interconnect with the switch input connection of the generator, and at least second and third connectors, the second and third connectors being adapted to interconnect respectively with the switch connectors of the first and second footswitch assemblies.

2. An electrosurgical system according to claim 1, further comprising a handswitch mounted on one of the electrosurgical instruments, the handswitch comprising a switch module, a cable and a switch connector, the adapter unit further comprising a fourth connector adapted to interconnect with the switch connector of the handswitch.

3. An electrosurgical system according to claim 2, wherein there are at least first and second electrosurgical assemblies respectively connected to first and second output connections of the generator.

4. An electrosurgical system according to claim 1, wherein the adapter unit comprises a cable unit, comprising only cables and connectors.

5. An electrosurgical system according to claim 4, wherein the adapter unit comprises a “Y-Cable”.

6. An electrosurgical system according to claim 4, wherein the adapter unit comprises an “X-Cable”.

7. An electrosurgical system comprising:
i) an electrosurgical generator comprising:
a) at least one source of radio frequency (RF) power,
b) at least two output connections, the output connections being such that at least two electrosurgical assemblies can be connected to the generator, and
c) a switch input connection;
ii) at least two electrosurgical assemblies, each comprising an electrosurgical instrument and a cable connecting the electrosurgical instrument to a respective output connection;
iii) at least first and second handswitch assemblies, each adapted to control the operation of the respective electrosurgical assembly, and each comprising a switch module, a cable and a switch connector; and
iv) an adapter unit comprising a first connector adapted to interconnect with the switch input connection of the generator, and at least second and third connectors, the second and third connectors being adapted to interconnect with the switch connectors of the first and second handswitch assemblies.

8. An electrosurgical system according to claim 7, wherein the adapter unit comprises a cable unit, comprising only cables and connectors.

9. An electrosurgical system according to claim 8, wherein the adapter unit comprises a “Y-Cable”.

10. An electrosurgical system according to claim 8, wherein the adapter unit comprises an “X-Cable”.
